(19) United States
(12) Patent Application Publication
(10) Pub. No.: US 2007/0134306 A1
(43) Pub. Date: Jun. 14, 2007

(54) METHOD AND DEVICE FOR ACCELERATING THE GROWTH OF SKIN APPENDAGES, IN PARTICULAR NAILS

(75) Inventors: Antoine KOYAZOUNDA, Montmeyran (FR); Jean-Claude MILLET, Etoile sur Rhone (FR)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: MILLET INNOVATION, Loriol sur Drome (FR)

(21) Appl. No.: 11/613,603

(22) Filed: Dec. 20, 2006

Related U.S. Application Data
(63) Continuation of application No. PCT/FR05/01514, filed on Jun. 17, 2005.

(30) Foreign Application Priority Data
Jun. 22, 2004 (FR) 0406744

(51) Int. Cl. A61L 15/00

(52) U.S. Cl. 424/445

(57) ABSTRACT

A method and a device are provided for accelerating the growth of skin appendages. According to one embodiment, a dressing is provided having an enveloping fabric and a lining material designed to be in contact with at least one appendage, and a liquid or viscous base containing water in a proportion sufficient for maintaining a high degree of moisture near the appendage. The liquid or viscous base is deposited on the lining material of the dressing, and the dressing is placed in such a way that the lining material is in contact with the appendage.

### CROSS-REFERENCE TO RELATED APPLICATIONS

This application is a Continuation of International Application No. PCT/FR2005/001514, filed Jun. 17, 2005, which was published in the French language on Jan. 26, 2006, under International Publication No. WO 2006/008373 A1 and the disclosure of which is incorporated herein by reference.

### BACKGROUND OF THE INVENTION

The present invention relates to a method for accelerating the growth of skin appendages and, more particularly, the growth of nails, as well as a device enabling this method to be implemented.

Appendages are visible epidermic productions of the human or animal body, which form nails, claws, hairs, wool, feathers and horns. These epidermic productions consist essentially of keratin, a protein that results from the transformation of the cells of the skin. In the case of nails, the keratin is produced by the cells of a matrix located at the nail groove. This matrix is extended by a bed on which the nail rests, this bed being delimited by the peri-ungual groove.

In practice, nail growth is estimated at an average speed of approximately 0.1 mm a day. It takes approximately 4 to 6 months to fully renew fingernails and approximately 12 to 18 months to fully renew toenails, the growth speed of which is slower than that of fingernails.

This time is excessively long, and it is not possible, for a healthy subject having for example one broken nail, to accelerate the re-growth of this nail.

Similarly, it is not possible to accelerate the re-growth of the nails of subjects affected by nail mycoses, which are very frequent complaints, the notable effect of which is to slow down or even stop the growth of nails. Indeed, these nail mycoses are treated locally by the application of creams, or by the consumption of tablets the active principle of which is conveyed by the blood stream to the location of the infection. However, these creams or tablets only have an effect on the mycosis itself and do not favor the re-growth of the nails.

### BRIEF SUMMARY OF THE INVENTION

Thus, the present invention aims to provide a method and a device enabling the growth of skin appendages, and particularly nails, to be accelerated.

This object is achieved by providing a method comprising the following steps: providing a dressing comprising an enveloping fabric and a lining material designed to be in contact with at least one appendage, preparing a liquid or viscous base comprising an antimicrobial or antimycotic agent, a compound favoring the penetration of the antimicrobial or antimycotic agent into the skin and/or the appendage, and water in a proportion sufficient for maintaining a high degree of moisture near the appendage, depositing the liquid or viscous base onto the lining material of the dressing, and placing the dressing comprising the liquid or viscous base in such a way that the lining material is in contact with the appendage.

The appendage(s) treated are thus kept in an environment characterized by a high degree of moisture. The presence of an antimicrobial or antimycotic agent and of a compound favoring the penetration of the antimicrobial or antimycotic agent into the skin or the appendages, and the degree of moisture surprisingly help to favor the growth of the skin appendages.

According to one embodiment, the liquid or viscous base comprises water in a proportion greater than 90% of the total weight of the liquid or viscous base.

According to another embodiment, the compound favoring the penetration of the antimicrobial or antimycotic agent into the skin and/or the appendage is polyethylene glycol.

According to a further embodiment, the polyethylene glycol has a molecular weight greater than or equal to 6,000, preferably on the order of 8,000.

According to a still further embodiment, the liquid or viscous base further comprises a polysaccharide.

According to another embodiment, the polysaccharide is a scleroglucan.

According to a further embodiment, the liquid or viscous base further comprises an alcohol.

According to a still further embodiment, the antimicrobial or antimycotic agent is thymol.

The present invention also relates to a device for accelerating the growth of skin appendages, in particular of nails, comprising a substantially water-proof dressing comprising an enveloping fabric, a lining material of the dressing designed to be in contact with at least one appendage, and a liquid or viscous base in contact with the lining material and comprising an antimicrobial or antimycotic agent, a compound favoring the penetration of the antimicrobial or antimycotic agent into the skin and/or the appendage, and water in a proportion sufficient for maintaining a high degree of moisture near the appendage.

According to one embodiment, the dressing is occlusive.

According to another embodiment, the dressing forms a fingerstall.

According to a further embodiment, the enveloping fabric is an elastane polyamide and the lining material is a silicone gel.

According to a still further embodiment, the liquid or viscous base comprises water in a proportion greater than 90% of the total weight of the liquid or viscous base.

According to an additional embodiment, the liquid or viscous base further comprises a polysaccharide.

According to another embodiment, the polysaccharide is a scleroglucan.

According to a further embodiment, the liquid or viscous base further comprises an alcohol.

According to a still further embodiment, the antimicrobial or antimycotic agent is thymol.

According to an additional embodiment, the compound favoring the penetration of the antimicrobial or antimycotic agent into the skin and/or the appendage is polyethylene glycol.

According to still another embodiment, the polyethylene glycol has a molecular weight greater than or equal to 6,000, preferably on the order of 8,000.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a schematic perspective view of a device according to the present invention, illustrating a method of application to the end of a finger or a toe.

### DETAILED DESCRIPTION OF THE INVENTION

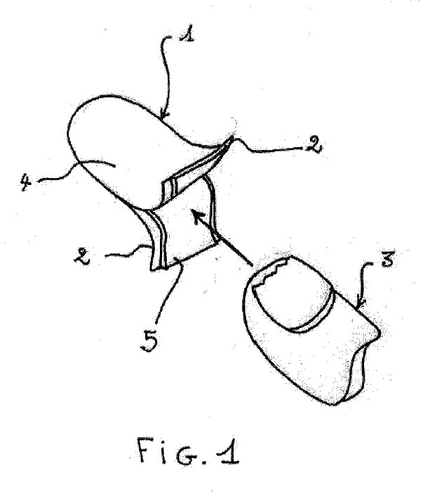

FIG. 1 schematically represents a device according to the present invention provided to be applied to the end of a finger or a toe, for example the big toe 3. The device here takes the shape of an occlusive dressing 1, substantially impermeable to water and other liquids, but possibly permitting gas exchanges depending on the embodiment chosen. The dressing here forms an enveloping fingerstall, that covers the entire last phalanx of the toe 3. This fingerstall is formed by a sheet folded and then sewn or welded along its edges over a portion of its length, or of two sheets superimposed then sewn or welded along their edges. The distal end of the fingerstall is not sewn or welded and thus has two flaps 2 that facilitate the positioning of the dressing on the toe 3 and can be cut so as to adapt the length of the fingerstall to that of the toe. The sewing or welding line or, more generally, the connecting line between the edges of the sheet or sheets forming the fingerstall, may not be totally impermeable to the liquid or viscous base described below, thus allowing gas exchanges.

Preferably, the sheet comprises an outer enveloping fabric 4 and an inner lining material 5. The fabric 4 comprises a mixture of elastic fibers and of thermoplastic fibers. The elastic fibers preferably comprise elastane and the thermoplastic fibers preferably comprise polyamide. The fabric comprises, for example, a mixture of polyamide and elastane fibers in respective proportions of 70% and 30% by weight.

The lining material 5 is designed to be directly in contact with the end of the finger or toe. It is for example formed by a silicone gel well known for its compatibility with skin. This is for example the silicone gel marketed by the applicant (Millet Innovation) under the brand name EpithElium 26™, which reproduces the characteristics of the footpad, and the gels marketed under the brand names EpithElium 27™ and EpithElium 27+™. These gels considerably limit the irritations that can result from friction in a humid environment.

The device according to the present invention comprises in addition a liquid or viscous base.

This liquid or viscous base is, for example, a gel or a cream. The base comprises an antimicrobial or antimycotic agent, a compound favoring the penetration of the antimicrobial or antimycotic agent into the skin and/or the nail, a polysaccharide, alcohol, and water in a significant proportion, preferably greater than 90% of the total weight of the base.

The antimicrobial agent is an antibacterial agent or an antimycotic agent. It is, for example, an antibacterial agent that can be of natural origin, for example thymol, the dissolution of which is favored by the presence of the alcohol in the base.

The compound favoring the penetration of the antimicrobial or antimycotic agent into the skin and/or the nail itself, softened by the presence of water, is, for example, polyethylene glycol, preferably a polyethylene glycol (PEG) of high molecular weight, greater than or equal to 6,000, preferably on the order of 8,000, that is very stable over time.

The polysaccharide is particularly a sclerotium gum obtained by fermentation of a mold, Sclerotium rolfsii, in a glucose-enriched environment. In the presence of water, this polysaccharide forms a transparent gel.

It will be understood that other compounds can be added to the liquid or viscous base according to the present invention. These are particularly compounds intended to provide the skin with nutriments, such as vitamin A.

### EXAMPLE

The liquid or viscous base can, for example, be produced in the following manner. First, 5 grams of polyethylene glycol of a molecular weight on the order of 8,000are mixed into 20 grams of alcohol, until a homogenous liquid forming a mixture A is obtained. Then, 1 gram of thymol is added to mixture A and is mixed until full dissolution to obtain a mixture B. Then, a polysaccharide solution is prepared, by mixing 0.8% by weight of sclerotium gum into water to obtain a mixture C. Finally, mixture B is mixed into mixture C according to the proportions 1 part by weight B to 99 parts by weight C, which results in a relatively liquid and transparent gel, constituting the liquid or viscous base.

To implement the present invention, a small knob of gel is placed in the bottom of the fingerstall, and this fingerstall is positioned on the toe in such a way that it covers the entire end of the toe.

Thus an environment is immediately created that is humid to saturation (in excess of 80% humidity), anti-infectious and nourishing for the nail. The fingerstall is worn for as long as possible according to the various constraints of the users, but no less than 12 hours per day. The environment that is humid, anti-infectious and nourishing for the nail is thus maintained over time.

It appears that the device according to the present invention produces an effect on the nail within a few days, in practice from 15 to 30 days, depending on the time for which the device is used every day. At the end of this period of time, a growth speed of the nail can be observed that is approximately three times greater than the speed observed in the usual circumstances for a healthy subject. This is a total breakthrough compared to what is known to date. In the specific case of a toe nail, it can be seen that the full renewal time of the nail according to the method of the present invention is reduced to approximately 4 months instead of 12 to 18 months for a healthy subject.

It will be understood that the dressing according to the present invention can be designed to receive several fingers or toes, for a collective application of the method to several nails.

It will be noted that exposure of the toes to a humid environment is deemed to favor the implantation and development of fungal infections responsible for mycoses. The present invention runs counter to this prejudice by creating a humid environment having no such disadvantage, even in the event that the subject has an onychomycosis. Moreover, in such a case, the onychomycosis gradually disappears by applying the device according to the present invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A device for accelerating growth of skin appendages, particularly nails, comprising:
a substantially water-proof dressing comprising an enveloping fabric,
a lining material for the dressing, the lining material designed to be in contact with at least one appendage,
a liquid or viscous base placed in the dressing in such a way that the liquid or viscous base will be in contact with the appendage upon application, the liquid or viscous base comprising:
an antimicrobial or antimycotic agent,
a compound favoring penetration of the antimicrobial or antimycotic agent into skin and/or the skin appendage,
a polysaccharide, and
water in a proportion sufficient for maintaining a high degree of moisture near the appendage to favor growth of the appendage.

2. The device according to claim 1, wherein the dressing is occlusive.

3. The device according to claim 1, wherein the dressing forms a fingerstall for growth of the nails.

4. The device according to claim 1, wherein the enveloping fabric comprises an elastane polyamide and the lining material comprises a silicone gel.

5. The device according to claim 1, wherein the liquid or viscous base comprises water in a proportion greater than 90% of a total weight of the liquid or viscous base.

6. The device according to claim 1, wherein the polysaccharide is a scleroglucan.

7. The device according to claim 1, wherein the liquid or viscous base further comprises an alcohol.

8. The device according to claim 1, wherein the antimicrobial or antimycotic agent comprises thymol.

9. The device according to claim 1, wherein the compound favoring penetration of the antimicrobial or antimycotic agent into the skin and/or the skin appendage comprises polyethylene glycol.

10. The device according to claim 9, wherein the polyethylene glycol has a molecular weight of at least 6,000.

11. A method for accelerating growth of skin appendages, particularly nails, the method comprising the following steps:
providing a dressing comprising an enveloping fabric and a lining material designed to be in contact with at least one appendage,
preparing a liquid or viscous base comprising an antimicrobial or antimycotic agent, a compound favoring penetration of the antimicrobial or antimycotic agent into skin and/or the skin appendage, and water in a proportion sufficient for maintaining a high degree of moisture near the appendage,
depositing the liquid or viscous base onto the lining material of the dressing, and
placing the dressing comprising the liquid or viscous base such that the lining material is in contact with the appendage.

12. The method according to claim 11, wherein the liquid or viscous base comprises water in a proportion greater than 90% of a total weight of the liquid or viscous base.

13. The method according to claim 11, wherein the compound favoring penetration of the antimicrobial or antimycotic agent into the skin and/or the skin appendage comprises polyethylene glycol.

14. The method according to claim 13, wherein the polyethylene glycol has a molecular weight of at least 6,000.

15. The method according to claim 11, wherein the liquid or viscous base further comprises a polysaccharide.

16. The method according to claim 15, wherein the polysaccharide comprises a scleroglucan.

17. The method according to claim 11, wherein the liquid or viscous base further comprises an alcohol.

18. The method according to claim 11, wherein the antimicrobial or antimycotic agent comprises thymol.

19. The device according to claim 10, wherein the molecular weight is on an order of 8000.

20. The method according to claim 14, wherein the molecular weight is on an order of 8000.
